(19) United States
(12) Patent Application Publication
(10) Pub. No.: US 2025/0195331 A1
(43) Pub. Date: Jun. 19, 2025

(54) THERMAL SENSOR FOR BIOPHARMECUTICAL STORAGE CONTAINERS AND METHODS OF CORRELATING THERMAL SENSORS

(71) Applicant: Sartorius Stedim North America Inc., Bohemia, NY (US)

(72) Inventors: Amy Grumet-Avallone, Smithtown, NY (US); Peter Smit, Mount Sinai, NY (US); Jake Duerwald, Smithtown, NY (US); Matthew Huhn, Queens, NY (US); Jonathan Cutting, Heber City, UT (US); Marc Sanchez, Arlington, MA (US); Matvey Sirotkin, Stony Brook, NY (US)

(73) Assignee: Sartorius Stedim North America Ins., Bohemia, NY (US)

(21) Appl. No.: 18/538,761

(22) Filed: Dec. 13, 2023

(51) Int. Cl. A61J 1/16; A61J 1/18

(52) U.S. Cl. CPC A61J 1/165; A61J 1/18; A61J 2200/72

(57) ABSTRACT

A storage container for aseptically storing and freezing a biopharmaceutical composition includes a frame, an expandable bladder, and a sensor system. The expandable bladder is supported within the frame having a filled state in which the expandable bladder is configured to hold the biopharmaceutical composition therein. The sensor system is secured to an exterior surface of the bladder. The sensor system is configured to transmit a signal indicative of a temperature of the exterior surface of the bladder which is correlated to a temperature of biopharmaceutical composition at a desired location within the expandable bladder in the filled state thereof.

### BACKGROUND

### 1. Technical Field

The present disclosure relates to thermal sensors and, more specifically, thermal sensors for biopharmaceutical storage containers.

### 2. Discussion of Related Art

Development of biopharmaceutical compositions includes developing protocols for evaluating the effects of freezing, thawing, heating, cooling, storing, or transferring biopharmaceutical compositions. The biopharmaceutical compositions may be monoclonal antibodies (mAbs), therapeutic proteins, vaccines, lipid nanoparticles, viruses, virus banks, exosomes, cell banks, and cell therapy products. The methods and protocols for freezing, thawing, heating, cooling, storing, or transferring of biopharmaceutical compositions may affect the formulation and/or the stability of the biopharmaceutical compositions. To generate protocols, samples of biopharmaceutical compositions must be evaluated through freezing, thawing, heating, cooling, storage, and transfer processes to analyze the effect of the processes on the biopharmaceutical compositions and the stability thereof.

### SUMMARY

This disclosure relates to thermal sensors for storage containers for biopharmaceutical compositions. The thermal sensors disclosed herein may be disposed within an interior of the storage container or may be disposed on the exterior of the storage container and are correlated to provide a temperature of the biopharmaceutical composition at a desired location within the storage container. The thermal sensor may be correlated to provide a temperature of the biopharmaceutical composition at the center of or some other desired location within the storage container.

In an embodiment of the present disclosure, a storage container for aseptically storing and freezing a biopharmaceutical composition includes a frame, an expandable bladder, and a sensor system. the expandable bladder is supported within the frame having a filled state in which the expandable bladder is configured to hold the biopharmaceutical composition therein. The sensor system is secured to an exterior surface of the bladder. The sensor system is configured to transmit a signal indicative of a temperature of the exterior surface of the bladder which is correlated to a temperature of biopharmaceutical composition at a desired location within the expandable bladder in the filled state thereof.

In embodiments, the sensor system is within the envelope of the container and its supporting structure when the expandable bladder is in the filled state thereof. The sensor system may be configured to remain secured to the expandable bladder during a freezing process, shipping, storage, and a thawing process of the expandable bladder.

In some embodiments, the sensor system includes a body and a sensor. The body is secured to the exterior of the storage container. The sensor is disposed between the body and the exterior surface of the bladder with the sensor in contact with the exterior surface of the bladder. The sensor is configured to determine a temperature of the exterior surface of the bladder to generate the signal indicative of the temperature of the exterior surface of the bladder. The sensor may be a resistance temperature sensor. The sensor may include a thermally conductive housing in contact with the exterior surface of the bladder. The sensor system may include a sensor cable that extends from the sensor and that terminates in a connector. The body of the sensor system may include a connector holder and a cable guide. The sensor cable may be received within the cable guide to secure the sensor cable relative to the body. The connector may be received within the connector holder to secure the connector relative to the body. The body may include attachment features that engage the bladder to releaseably secure the body to the bladder.

In another embodiment of the present disclosure, a system for determining a temperature of a biopharmaceutical composition during a freezing process that includes a storage container, a sensor assembly, and a processing device. The storage container is configured to aseptically store and freeze a biopharmaceutical compositing. The storage container includes a frame and an expandable bladder. The expandable bladder is supported within the frame and has a filed state in which the expandable bladder is configured to hold the biopharmaceutical composition therein. The sensor system is secured to an exterior surface of the bladder and is configured to transmit a signal indicative of a temperature of the exterior surface of the bladder. The processing device is configured to receive the signal from the sensor system. The processing device provides a temperature of the biopharmaceutical composition at a desired location within the bladder based on the temperature of the exterior surface of the bladder.

In embodiments, the system includes a probe system that includes a probe disposed within an interior of the expandable bladder. The probe may be configured to directly measure and transmit a signal indicative of a temperature at the desired location within the bladder. The processing device may be configured to correlate the signal provided by the senor system to the temperature of the biopharmaceutical composition at the desired location within the bladder based on the signal of the probe.

In another embodiment of the present disclosure, a method of correlating an external sensor system for a storage container filled with a biopharmaceutical composition includes freezing the storage container filled with the biopharmaceutical composition. The method includes generating a first temperature signal that is indicative of a temperature of an exterior surface of the storage container with the external sensor system that is secured to the exterior surface of the storage container during freezing of the storage container. The method includes generating a second temperature signal indicative of a temperature of the biopharmaceutical composition within the storage container with an internal probe system that is disposed within an interior of the storage container n direct contact with the biopharmaceutical composition during freezing of the storage container. The method includes receiving the first temperature signal and the second temperature signal with a processing device and generate correlated external temperature data to correlate the temperature of the exterior surface of the storage container to a temperature of the biopharmaceutical composition within the interior of the storage container based on the first temperature signal and the second temperature signal.

In embodiments, the method includes freezing another storage container that is filled with the biopharmaceutical composition and determining a temperature of the biopharmaceutical composition within the other storage container with only an external sensor system using the correlated external temperature data.

In some embodiments, determining the temperature of the biopharmaceutical composition within the other storage container includes looking up the temperature of the biopharmaceutical composition in a look up table of the correlated external temperature data. Determining the temperature of the biopharmaceutical composition within the other storage container includes calculating the temperature of the biopharmaceutical composition using a correlation algorithm of the correlated external temperature data. The method may include shipping the other storage container with the external sensor system that is secured to the storage container. The external sensor system may be disposed within extremities of a frame of the other storage container.

In certain embodiments, generating the second temperature signal includes the internal probe system that is disposed at a desired location within the interior of the storage container. The method may include inserting a probe of the probe system into an interior of the storage container such that a tip of the probe is positioned at a desired location within the interior of the storage container.

In another embodiment of the present disclosure, a method of correlating an external sensor system for a storage container that is filled with a biopharmaceutical composition includes freezing the storage container filled with the biopharmaceutical composition. The method includes generating a temperature signal indicative of a temperature of an exterior surface of the storage container with the external sensor system that is secured to the exterior surface of the storage container during freezing of the storage container. The method includes determining a temperature of the biopharmaceutical composition at a desired location within the storage container using the temperature signal and the correlated external temperature data.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a storage container including thermal sensor systems provided in accordance with embodiments of the present disclosure;

FIG. 2 is a schematic view of a bladder of the storage container of FIG. 1;

FIG. 3 is a top view of the storage container of FIG. 1;

FIG. 4 is a bottom perspective view of a thermal sensor assembly of the thermal sensor system of FIG. 3;

FIG. 5 is a top perspective view of the thermal sensor assembly of FIG. 4;

FIG. 6 is a perspective view of another thermal sensor assembly of the storage container of FIG. 1;

FIG. 7 is a flowchart of a method of correlating a thermal sensor system in accordance with embodiments of the present disclosure;

FIG. 8 is a graph of example external and internal temperatures of contents of a storage container over time during a freezing process;

FIG. 9 is a flowchart of a method of determining a temperature of the contents of a storage container with an external sensor system provided in accordance with embodiments of the present disclosure; and

FIG. 10 is a block diagram of an example system that may perform one or more of the operations described herein in accordance with embodiments of the present disclosure.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like. Further, as used herein the term “biopharmaceutical compositions” refers to a product coming from biotechnology, culture environments, cell cultures, buffer solutions, artificial nutrition liquids, blood products and derivatives of blood products, a pharmaceutical product, or more generally a product intended to be used in the medical field including, without any limitation, monoclonal antibodies (mAbs), therapeutic proteins, viruses, lipid nanoparticles, vaccines, virus banks, exosomes, cell banks, and cell therapy products. As used herein, the term “cryogenic” refers to temperatures in a range of −20° C. to −196° C. unless otherwise specified.

This application is directed to thermal sensors for storage containers for biopharmaceutical compositions. The thermals sensors disclosed herein may be disposed within an interior of the storage container or may be disposed on an outer surface of the storage container on the exterior of the storage container. The thermal sensors disposed on the exterior of the storage container may be correlated to provide a temperature of the biopharmaceutical composition within the storage container. For example, the thermal sensor disposed on the exterior of the storage container may be correlated to provide a temperature of the biopharmaceutical composition at the center, or some other desired location, of within the storage container. The application may include a method of correlating a thermal sensor on the exterior of the storage container. It should be appreciated that it may be convenient to measure an exterior temperature over the inconvenience to directly measure an interior temperature. For example, the inconvenience to directly measure internal temperatures may include accurately positioning an internal sensor in a flexible bladder, may include a risk of damage to the flexible bladder during sensor insertion/removal which may result in leaking and/or contamination, may include high insertion forces to overcome hydrostatic forces to insert a sensor into a thermowell, and/or may include high removal forces to remove a sensor from a thermowell once frozen.

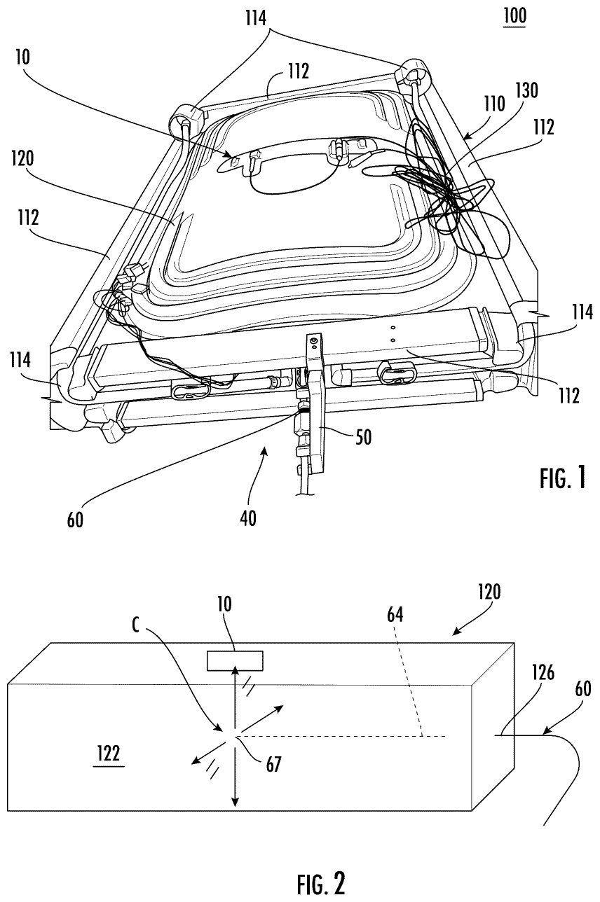

Referring now to FIG. 1, a storage container 100 provided in accordance with the present disclosure. The storage container 100 is designed to receive a biopharmaceutical composition and hold the biopharmaceutical composition through freezing, storage, shipping, thawing, and distribution. The storage container 100 may be aseptic or sterile depending on the biopharmaceutical composition being stored therein. The storage container 100 may include a frame 110 and a bladder 120. The frame 110 may be formed of a rigid material to contain and protect the bladder 120. The frame 110 may form a rectangular enclosure to hold the bladder 120 therein.

The frame 110 may include side rails 112 and corner elements 114 that join the side rails 112 together. The corner elements 114 may be of a material configured to absorb force without deforming. For example, the corner elements 114 may be formed of a plastic, e.g., a thermoset or a thermoplastic, that can impact another storage container 100 or structure without being damaged or breaking. The corner elements 114 may be configured to function at cryogenic temperatures. In some embodiments, the corner elements 114 may be formed of a metal such as aluminum or stainless steel. The side rails 112 may be formed of a metal such as aluminum or stainless steel. In some embodiments, the side rails 112 may be formed of plastic material. The frame 110 is stackable with other frames 110 such that multiple storage containers 100 can be stacked on top of one another.

With additional reference to FIG. 2, the bladder 120 is disposed within the frame 110. The frame 110 supporting the bladder 120 within the frame 110 and protecting the bladder 120. The bladder 120 is formed of a flexible material that expands and collapses in response to being filled and drained, respectively. For example, when the bladder 120 is empty, the bladder 120 may be collapsed with a minimal amount of air or gas in an interior 122 of the bladder 120. As the storage container 100 and thus, the bladder 120, is filled with a biopharmaceutical composition, the bladder 120 expands. When the bladder 120 is filled, the frame 110 maintains the bladder 120 in a substantially rectangular prismatic shape.

During freezing, storage, and thawing of a biopharmaceutical composition within the storage container 100, it may be advantageous to know the temperature of the biopharmaceutical composition within the bladder 120 of the storage container 100. Specifically, as a biopharmaceutical composition is frozen or thawed, the biopharmaceutical composition may freeze or thaw at different rates depending on where in the bladder 120 the temperature is being measured. For example, portions of a biopharmaceutical composition adjacent the extremities or outside edges of the bladder 120 may change temperature more quickly than portions of the biopharmaceutical composition at or adjacent the center “C” of the bladder 120. The different rates may be based on conduction and/or convection within the interior 122 of the bladder 120 and may affect the rate at which the biopharmaceutical composition changes temperature. In embodiments, properties of the biopharmaceutical composition itself may affect the rate at which the biopharmaceutical composition changes temperature.

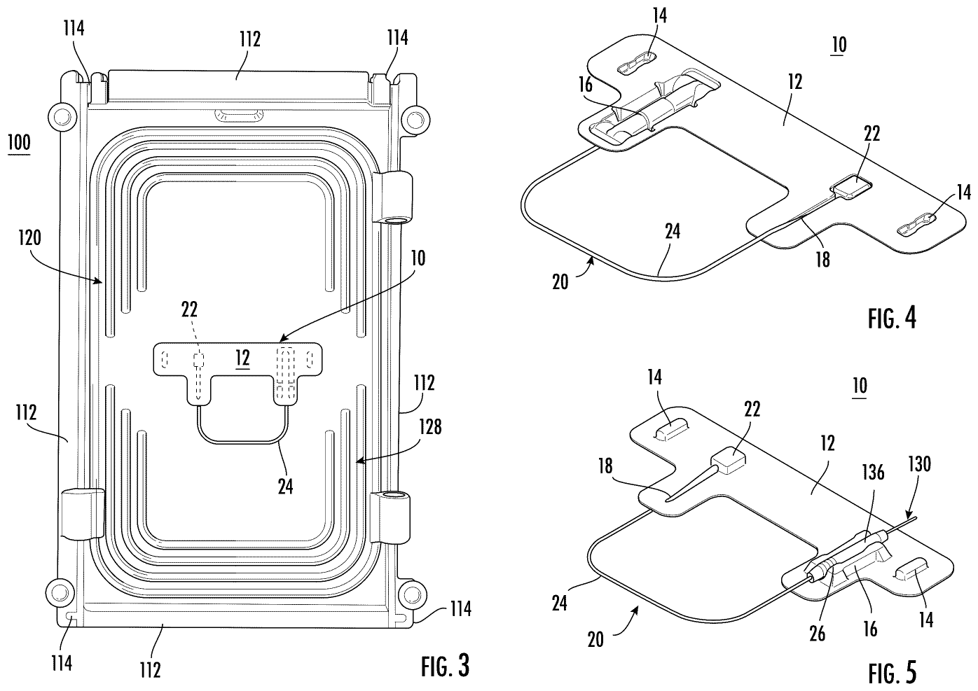

Referring now to FIGS. 3-5, an external thermal sensor system is described in accordance with the present disclosure and is referred to generally as sensor system 10. The sensor system 10 is attachable to the outside surface of the storage container 100 and is configured to monitor a temperature of the contents of the storage container 100. The sensor system 10 is configured to generate and transmit a signal indicative of a temperature of the exterior surface of the storage container 100. The sensor system 10 disposed within extremities of the frame 110. For example, in at least one embodiment, when the storage container 100 is empty. the sensor system 10 is disposed entirely within the envelope of the storage container 100 and its supporting structure. The sensor system 10 includes a body 12 and a sensor assembly 20. The body 12 is formed of rigid or semi-rigid material and is configured to secure and position the sensor assembly 20 on the storage container 100.

The body 12 includes attachment features 14 that engage the storage container 100 to secure the sensor system 10 to the storage container 100 and position the sensor assembly 20 against the exterior surface of the bladder 120. In some embodiments, the storage container 100 includes features that are engaged by the attachment features 14 to secure the sensor assembly 20 to the storage container 100. In particular embodiments, the bladder 120 of the storage container 100 includes features that are engaged by the attachment features 14. In certain embodiments, the attachment features 14 may each engage a side rail 112 to secure the sensor assembly 20 to the storage container 100. In some embodiments, the attachment features 14 include adhesive that engages an outer surface of the bladder 120 to secure the sensor assembly 20 to the storage container 100.

The body 12 may include a connector holder 16 and may include a cable guide 18. The connector holder 16 is sized and dimensioned to receive and hold a connector 26 of the sensor assembly 20. The cable guide 18 may be sized and positioned to receive and support a sensor cable 24 of the sensor assembly 20. The connector holder 16 and the cable guide 18 may be positioned on opposite sides of the body and may each be between the attachment features 14.

The sensor assembly 20 includes a sensor 22, the sensor cable 24, and the connector 26. The sensor 22 is configured to contact an exterior surface of the bladder 120. The sensor 22 may be a resistance temperature detector (RTD), a heat flux sensor (HFS), thermocouple, thermistor, bandgap temperature sensor, other temperature sensors known in the art, or combinations thereof. In at least one embodiment, an RTD, when implemented, and without being bound by any particular theory, is believed to provide the advantages of high accuracy and low drift. The sensor 22 may be disposed within a thermally conductive housing to protect the sensor 22 and/or to improve thermal transfer from the bladder 120 to the sensor 22. The sensor 22 may include a thermal transfer layer to contact the exterior surface of the bladder 120 to improve thermal transfer from the bladder 120 to the sensor 22. For example, the sensor 22 may include a thermal paste or a thermal conductive layer to contact the exterior surface of the bladder 120. In some embodiments, the bladder 120 may include a thermal transfer element bonded to the exterior surface of the bladder 120 to improve thermal transfer to the sensor 22. The sensor 22 is configured to measure a temperature of the bladder 120 and thus, the contents of the interior of the bladder 120. It is understood that the temperature at the exterior surface of the bladder 120 may differ from a temperature of the contents of the interior of the bladder 120; however, a correlation of the temperature at the exterior surface of the bladder 120 and the temperature of the contents of the interior of the bladder 120 will be discussed in detail below.

The sensor cable 24 extends from the sensor 22 to the connector 26 which terminates the sensor cable 24. The sensor cable 24 may pass through the cable guide 18 to position the sensor 22 relative to the body 12. The sensor cable 24 may also pass through a portion of the connector holder 16 such that the connector 26 is received and secured in the connector holder 16. The connector 26 is configured to receive and secure a connector 136 of a cable 130 that is configured to connect the sensor 22 to one or more devices that receive temperature data from the sensor 22. The cable 130 may connect to a wireless transmitter to transmit the temperature data to devices which may be local to or remote from the storage container 100 or shipping container housing the storage container 100.

The sensor system 10 may be a low-profile sensor system 10 that can be secured to the storage container 100 during filling, freezing, transport, storage, thawing, and distribution.

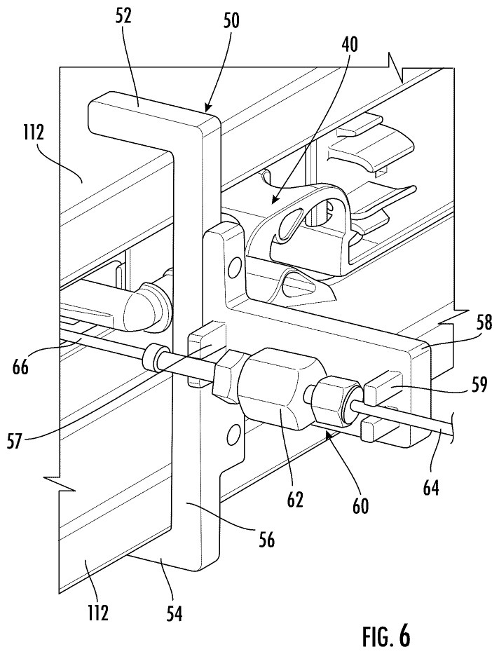

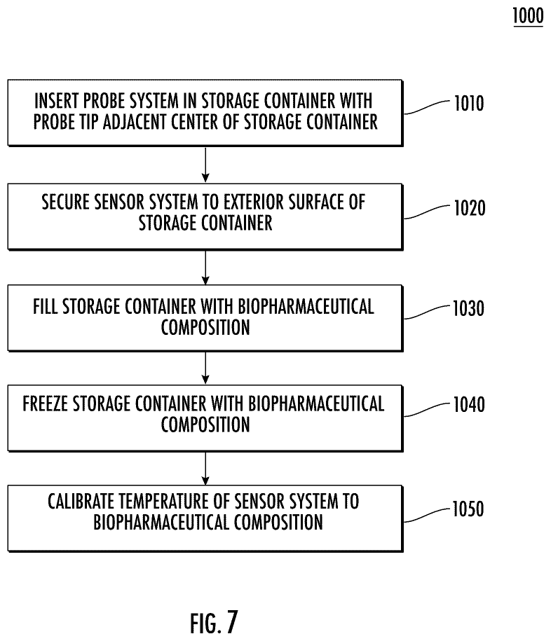

With reference to FIG. 6, an internal thermal probe system is described in accordance with the present disclosure and is referred to generally as probe system 40. The probe system 40 is attachable to the storage container 100 and directly measures the temperature of contents of the storage container 100. The probe system 40 includes a support 50 and a probe assembly 60. The support 50 mounts to a side rail 112 of the storage container 100 to position and secure the probe assembly 60 relative to the storage container 100. The support includes a top clamp 52 and a bottom clamp 54 that secure to a top and a bottom of the side rail 112, respectively. The top and bottom clamps 52, 54 may secure to the side rail 112 with one or more fasteners. The support 50 includes a backspan 56 and a brace 58. The backspan 56 extends between the top clamp 52 and the bottom clamp 54 and the brace 58 extends from the backspan 56 in a direction away from the storage container 100. The brace 58 may extend perpendicular from the backspan 56. The brace 58 includes a front mount 57 and a back mount 59 that are configured to receive and secure the probe assembly 60.

The probe assembly 60 includes a radial clamp 62, a probe guide 64, and a probe 66 having a tip 67. The probe guide 64 passes through an opening 126 in the bladder 120 (FIG. 2) such that the probe guide 64 is disposed in the interior 122 of the bladder 120. The opening 126 may seal about the exterior of the probe guide 64 to prevent leakage of the contents of the interior 122 of the bladder 120 and/or to prevent contamination of the contents of the bladder 120. The probe 66 passes through the probe guide 64 such that the tip 67 of the probe 66 is positioned at or adjacent the center of the bladder 120. The probe 66 is a sensor capable of directly measuring a temperature of the contents of the bladder 120 at the tip 67. The probe 66 may be separate from the probe guide 64 with the probe 66 sealing a passage of the probe guide 64 to prevent contents of the bladder 120 from leaking through the probe guide 64 or being contaminated through the probe guide 64. In some embodiments, the probe 66 and the probe guide 64 may be a single element. Portions of the probe 66 and the probe guide 64 disposed within the interior 122 of the bladder 120 may be sterilized such that the interior 122 of the bladder 120 remains aseptic or sterilized.

The probe 66 and the probe guide 64 pass through the radial clamp 62 with the radial clamp 62 having a locked configuration in which the probe guide 64 and the probe 66 are longitudinally fixed relative to the radial clamp 62 and an unlocked configuration in which the probe guide 64 and/or the probe 66 are longitudinally moveable relative to the radial clamp 62. The radial clamp 62 and/or the probe guide 64 are secured in the mounts 57, 59 of the brace 58 to secure the radial clamp 62 and thus, the probe 66, to the bladder 120.

It should be appreciated that aspects of the invention allow for the use of a correlation between the temperature at two points which are located on the exterior, within the interior of a container, or one located on the exterior and one located within the interior of a container. Without being bound by any particular theory, by measuring at a first point and then applying the correlation, the temperature at the second point can be estimated.

It should further be appreciated that the correlation may be established by taking temperature measurements of a first point and of a second point, but there are also soft sensors which employ a mechanistic model which may incorporate geometry and physical equations, usually in the form of differential equations. Thus in at least one embodiment, a soft sensor approach is used to improve the accuracy of the correlation made by measuring the temperature at two locations. In such embodiments, the incorporation of soft sensors may make it unnecessary to measure the temperature at two points.

Referring now to FIG. 7, a method of correlating an external thermal probe system with an internal thermal probe system for a pharmaceutical composition is described in accordance with the present disclosure and is referred to generally as method 1000 and with reference to the storage container 100, the sensor system 10, and the probe system 40 of FIGS. 1-6. The method 1000 may include inserting an internal probe system 40 into a bladder 120 of the storage container 100 (Step 1010). Inserting the internal probe system 40 may include securing a support 50 to a side rail 112 of the storage container 100. With the support 50 secured to the side rail 112, a probe 66 may be inserted through an opening 126 in the bladder 120 such that the probe 66 is disposed within the interior 122 of the bladder 120. The opening 126 may include a septum to sealingly receive the probe 66 therethrough. In some embodiments, the probe 66 is inserted through a probe guide 64 that is inserted through the opening 126. With the probe 66 disposed within the interior 122 of the bladder 120, a radial clamp 62 is secured to a brace 58 of the support 50 with the probe 66 disposed therethrough. The tip 67 of the probe 66 is positioned adjacent the center “C” of the bladder 120 and the radial clamp 62 is transitioned to a locked configuration such that the tip 67 is secured adjacent the center “C” of the bladder 120. The probe 66 may be place in signal communication with a processing device 1202 by attaching a cable extending from the probe 66 to the processing device 1202. The processing device 1202 may receive signals from the probe 66 indicative of a temperature at the tip 67 of the probe 66. The processing device 1202 may transmit data indicative of the temperature at the tip 67 of the probe 66 to one or more other devices. The processing device 1202 may transmit data wirelessly to one or more other devices.

The method 1000 may include securing an external sensor system 10 to an exterior surface of the bladder 120 (Step 1020). Securing the external sensor system 10 to the bladder 120 may include engaging attachment features 14 of the sensor system 10 with the bladder 120. When the attachment features 14 are engaged with the bladder 120, the sensor 22 is in contact with the exterior surface of the bladder 120. The sensor 22 provides a signal indicative of the temperature of the exterior surface of the bladder 120. The method 1000 may include securing a cable 130 to the sensor system 10 such that the sensor system 10 provides a signal indicative of the temperature of the exterior surface of the bladder 120 to a processing device 1202. The processing device 1202 may transmit data indicative of the temperature of the exterior surface of the bladder 120 to one or more other devices. The processing device 1202 may transmit data wirelessly to one or more other devices. The processing device 1202 may be the same processing device 1202 which receive signals indicative of the temperature of the tip 67 of the probe 66.

The method 1000 may include filling the bladder 120 of the storage container 100 with a biopharmaceutical composition (Step 1030). When the bladder 120 is filled with the biopharmaceutical composition, the storage container 100 is placed in a freezing device to freeze the biopharmaceutical composition in the bladder 120 (Step 1040). As the biopharmaceutical composition is frozen, the temperature of the tip 67 of the probe 66 and the temperature of the exterior surface of the bladder 120 are recorded. The temperatures may be recorded by the processing device 1202. The temperatures may be recorded until the temperature of the tip 67 of the probe 66 is at a desired temperature. The desired temperature may be a cryogenic temperature.

The method 1000 includes correlating a temperature of the exterior surface of the bladder 120 with a temperature of the biopharmaceutical composition at the center “C” of the bladder 120 (Operation 1050), or at some other desired location within the bladder 120. The temperature at the tip 67 of the probe 66 is substantially equal to the temperature of a biopharmaceutical composition at the center “C” of the bladder 120, or at some other desired location within the bladder 120. The temperature at the tip 67 of the probe 66 may be slightly different from the temperature of the biopharmaceutical composition at the center “C” of the bladder 120), or at some other desired location within the bladder 120. In some embodiments, there may be some conduction along the probe 66 such that the temperature at the tip 67 is slightly lower than the temperature of the biopharmaceutical composition at the center “C” of the bladder 120, or at some other desired location within the bladder 120. The body of the probe 66 and/or the probe guide 64 may be formed of a non-thermally conductive or a low thermally conductive material to minimize conduction along the probe 66 affecting the temperature of the tip 67 of the probe 66.

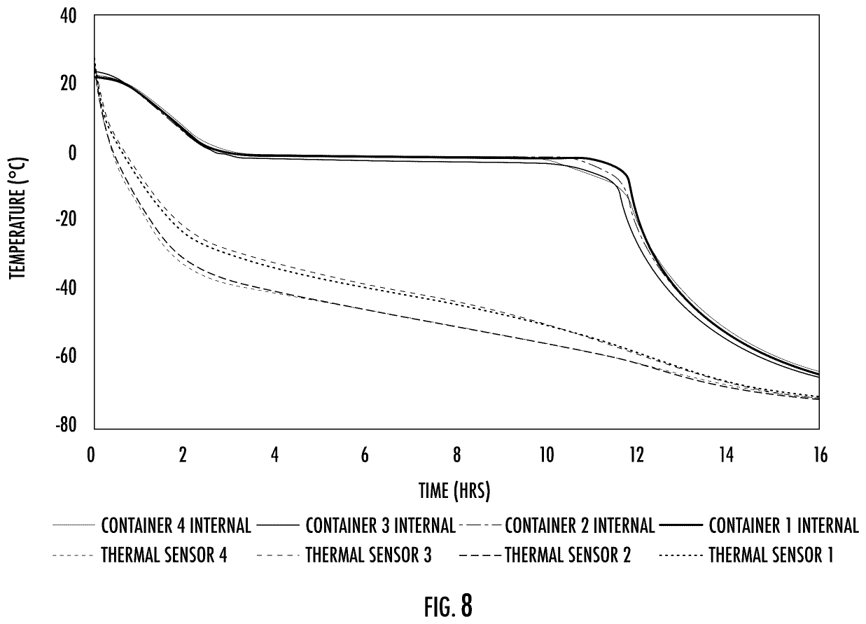

With particular reference to FIG. 8, the temperatures of the exterior surface of respective bladder 120 for separate storage containers and the temperature of a tip 67 of a respective probe 66 disposed within storage container 100 is shown during an example freezing process for a given biopharmaceutical composition within the storage containers 100. As shown, the internal and external temperatures both decrease quickly for the first two hours with the external temperatures decreasing at a greater rate than the internal temperature. Then from hours 2 to 14, the external temperatures decrease at a steady rate. In contrast, the internal temperatures remain relatively constant between hour 2 until hour 11 at which the internal temperature declines quickly until the temperature difference or delta with the external temperature is minimal. A minimal temperature delta may be in a range of 5 degrees Celsius to 10 degrees Celsius.

The operation 1050 may include developing a correlation algorithm or look up table to correlate the temperature of the external temperature of the bladder 120 over time with the temperature at the center of the bladder 120, or at some other desired location within the bladder 120. The correlation algorithm or look up table may be specific for a given biopharmaceutical composition such that a correlation algorithm may be required for each biopharmaceutical composition as detailed above with respect to method 1000. The correlation algorithms may be used to correlate the external temperature of the bladder 120 as measured by the sensor system 10 to a temperature at the center of the bladder 120, or at some other desired location within the bladder 120, when filled with a known biopharmaceutical composition. As such, the temperature at the center of the bladder 120, or at some other desired location within the bladder 120, may be determined without the need for the internal probe system 40. Thus, once the correlation algorithm is developed for a biopharmaceutical composition, the storage containers 100 may be used with only the external sensor system 10. Further, with the correlation algorithm, the bladder 120 may be provided without the opening 126. A bladder 120 without the probe system 40 and/or the opening 126 may have a decreased risk of contamination of a biopharmaceutical composition within the bladder 120. In some instances, a filled bladder 120 may have an air bubble at the top of the bladder 120 adjacent the sensor system 10. In such instances it has been found that the air bubble does not significantly change the correlation of the sensor system 10.

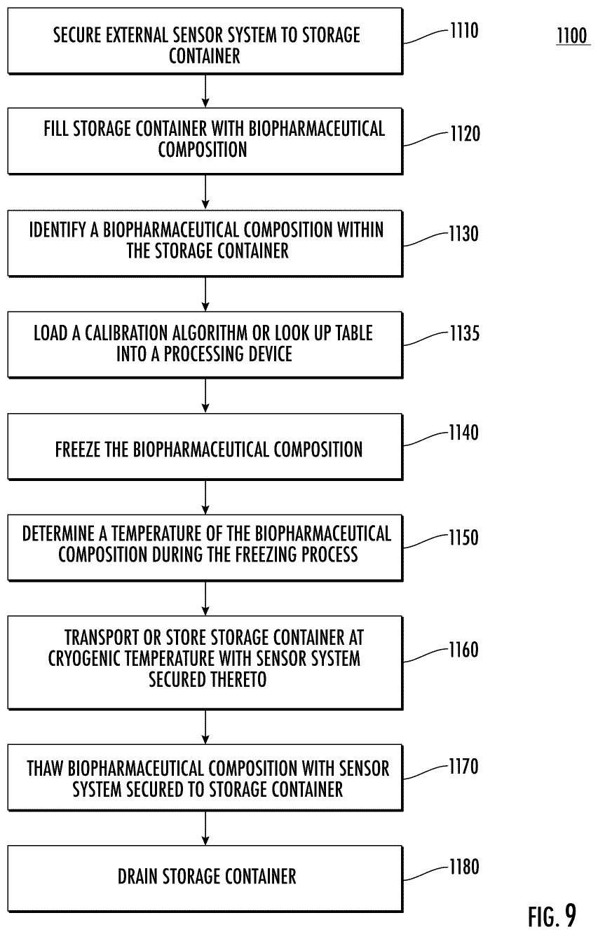

Referring now to FIG. 9, a method of determining a temperature of a biopharmaceutical composition within a storage container is disclosed in accordance with the present disclosure and is referred to generally as method 1100 with reference to the storage container 100 of FIGS. 1-5. The method 1100 may include securing an external sensor system 10 to an exterior surface of the bladder 120 (Step 1110). Securing the external sensor system 10 to the bladder 120 may include engaging attachment features 14 of the sensor system 10 with the bladder 120. When the attachment features 14 are engaged with the bladder 120, the sensor 22 is in contact with the exterior surface of the bladder 120. The sensor 22 provides a signal indicative of the temperature of the exterior surface of the bladder 120. The method 1100 may include securing a cable 130 to the sensor system 10 such that the sensor system 10 provides a signal indicative of the temperature of the exterior surface of the bladder 120 to a processing device 1202. The processing device 1202 may transmit data indicative of the temperature of the exterior surface of the bladder 120 to one or more other devices. The processing device 1202 may transmit data wirelessly to one or more other devices. The processing device 1202 may be the same processing device 1202 which receive signals indicative of the temperature of the tip 67 of the probe 66.

The method 1100 may include filling the bladder 120 of the storage container 100 with a biopharmaceutical composition (Step 1120). The method 1100 may include identifying a biopharmaceutical composition within the storage container 100 in a processing device 1202 (Step 1130). Identifying the biopharmaceutical composition may cause a correlation algorithm or a look up table for a freezing process of the biopharmaceutical composition to be loaded into the processing device 1202 (Step 1135). When the bladder 120 is filled with the biopharmaceutical composition and the biopharmaceutical composition identified, the storage container 100 is placed in a freezing device to freeze the biopharmaceutical composition in the bladder 120 (Step 1140). As the biopharmaceutical composition is frozen, the temperature of the exterior surface of the bladder 120 are transmitted to the processing device 1202 which uses external temperature and the correlation algorithm or look up table to determine a temperature of the biopharmaceutical composition at the center of the bladder 120 (Step 1150), or at some other desired location within the bladder 120. The freezing process may be continued until the temperature of the biopharmaceutical composition is at a desired temperature.

With the biopharmaceutical composition at a desired temperature, the storage container 100 may be transported and/or stored at cryogenic temperatures (Step 1160). The storage container 100 may be transported or stored with the sensor system 10 secured to the storage container 100. The sensor system 10 may be used to monitor a temperature of the biopharmaceutical composition during transportation and/or storage. In some embodiments, the sensor system 10 may be removed from the storage container 100 after the freezing process.

The method 1100 may include thawing the biopharmaceutical composition with the sensor system 10 secured to the storage container 100 (Step 1170). The method 1100 may include draining the storage container 100 with the sensor system 10 secured to the storage container (Step 1180). In some embodiments, the sensor system 10 is removed from the storage container 100 after the storage container 100 is drained and secured to another storage container 100 such that the sensor system 10 is reused.

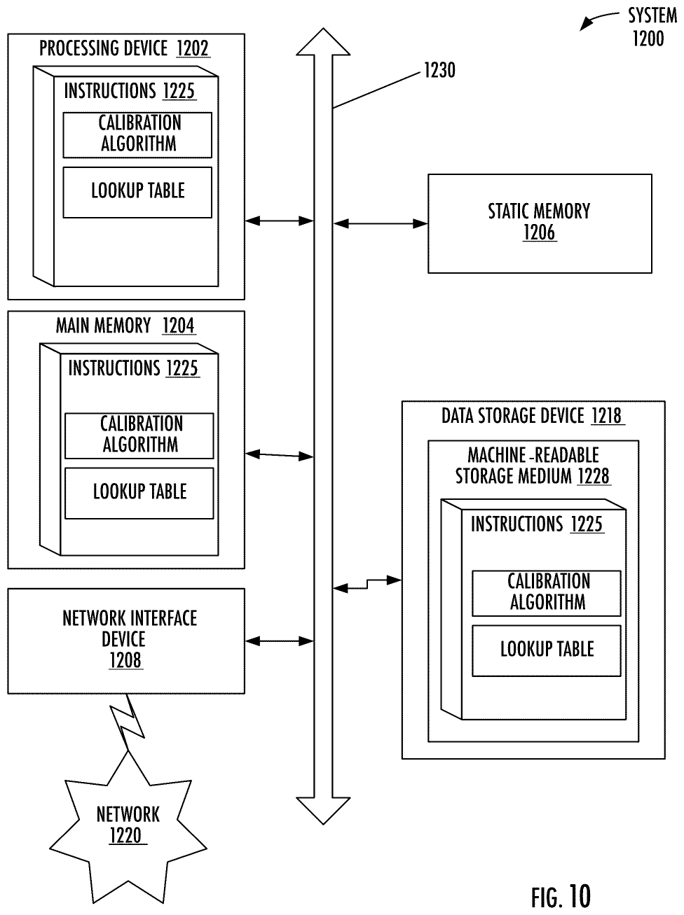

FIG. 10 is a block diagram of an example computing device 1200 that may perform one or more of the operations described herein, in accordance with some embodiments. Computing device 1200 may be connected to other computing devices in a LAN, an intranet, an extranet, and/or the Internet. The computing device may operate in the capacity of a server machine in a client-server network environment or in the capacity of a client in a peer-to-peer network environment. The computing device may be provided by a personal computer (PC), a Programmable Logic Controller (PLC), set-top box (STB), a server, a network router, switch or bridge, or any machine capable of monitoring or controlling a process, or executing a set of instructions (sequential or otherwise) that specify actions to be taken by that machine. In some embodiments, while only a single computing device is illustrated, the term “computing device” may be taken to include any collection of computing devices that individually or jointly execute a set (or multiple sets) of instructions to perform the methods discussed herein.

The example computing device 1200 may include a processing device (e.g., a general-purpose processor, a PLD, etc.) 1202, a main memory 1204 (e.g., synchronous dynamic random-access memory (DRAM), read-only memory (ROM)), a static memory 1206 (e.g., flash memory and a data storage device 1218), which may communicate with each other via a bus 1230.

Processing device 1202 may be provided by one or more general-purpose processing devices such as a microprocessor, central processing unit, or the like. In an illustrative example, processing device 1202 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1202 may comprise one or more special-purpose processing devices such as an application specific integrated circuit (ASIC), a field programmable gate array (FPGA), a digital signal processor (DSP), network processor, or the like. The processing device 1202 may be configured to execute the operations described herein, in accordance with one or more aspects of the present disclosure, for performing the operations and steps discussed herein.

Computing device 1200 may include a network interface device 1208 which may communicate with a communication network 1220. The computing device 1200 may include a video display unit (e.g., a liquid crystal display (LCD) or a cathode ray tube (CRT)), an alphanumeric input device (e.g., a keyboard), a cursor control device (e.g., a mouse) and an acoustic signal generation device (e.g., a speaker). In one embodiment, a video display unit, alphanumeric input device, and cursor control device may be combined into a single component or device (e.g., an LCD touch screen).

Data storage device 1218 may include a computer-readable storage medium 1228 on which may be stored one or more sets of instructions 1225 that may include instructions for one or more components (e.g., correlation algorithm or look up table) for carrying out the operations described herein, in accordance with one or more aspects of the present disclosure. Instructions 1225 may reside, completely or at least partially, within main memory 1204 and/or within processing device 1202 during execution thereof by computing device 1200, main memory 1204, and processing device 1202 constituting computer-readable media. The instructions 1225 may be transmitted or received over a communication network 1220 via network interface device 1208.

While computer-readable storage medium 1228 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” may be taken to include any medium that is capable of storing, encoding, or carrying a set of instructions for execution by the machine and that cause the machine to perform the methods described herein. The term “computer-readable storage medium” shall accordingly be taken to include, but not be limited to, solid-state memories, optical media, and magnetic media.

Examples described herein may relate to an apparatus for performing the operations described herein. This apparatus may be specially constructed for the required purposes, or it may comprise a general-purpose computing device selectively programmed by a computer program stored in the computing device. Such a computer program may be stored in a computer-readable non-transitory storage medium.

The methods and illustrative examples described herein are not inherently related to any particular computer or other apparatus. Various general-purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

Although the method operations were described in a specific order, it should be understood that other operations may be performed in between described operations, described operations may be adjusted so that they occur at slightly different times, or the described operations may be distributed in a system which allows the occurrence of the processing operations at various intervals associated with the processing.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A storage container for aseptically storing and freezing a biopharmaceutical composition, the storage container comprising:
a frame;
an expandable bladder supported within the frame, the expandable bladder having a filled state in which the expandable bladder is configured to hold the biopharmaceutical composition therein; and
a sensor system secured to an exterior surface of the bladder, the sensor system configured to transmit a signal indicative of a temperature of the exterior surface of the bladder which is correlated to a temperature of biopharmaceutical composition at a desired location within the expandable bladder in the filled state thereof.

2. The storage container according to claim 1, wherein the sensor system is within the envelope of the container and its supporting structure when the expandable bladder is in the filled state thereof.

3. The storage container according to claim 1, wherein the sensor system is configured to remain secured to the expandable bladder during a freezing process, shipping, storage, and a thawing process of the expandable bladder.

4. The storage container according to claim 1, where the sensor system comprises:
a body secured to the exterior of the storage container; and
a sensor disposed between the body and the exterior surface of the bladder with the sensor in contact with the exterior surface of the bladder, the sensor configured to determine a temperature of the exterior surface of the bladder to generate the signal indicative of the temperature of the exterior surface of the bladder.

5. The storage container according to claim 4, wherein the sensor is a resistance temperature detector.

6. The storage container according to claim 4, wherein the sensor includes a thermally conductive housing in contact with the exterior surface of the bladder.

7. The storage container according to claim 4, wherein the sensor system further comprises a sensor cable that extends from the sensor and terminates in a connector.

8. The storage container according to claim 7, wherein the body of the sensor system includes a connector holder and a cable guide, the sensor cable received within cable guide to secure the sensor cable relative to the body, the connector received within the connector holder to secure the connector relative to the body.

9. The storage container according to claim 4, wherein the body includes attachment features that engage the bladder to releaseably secure the body to the bladder.

10. A system for determining a temperature of a biopharmaceutical composition during a freezing process, the system comprising:
a storage container for aseptically storing and freezing a biopharmaceutical composition, the storage container comprising:
a frame; and
an expandable bladder supported within the frame, the expandable bladder having a filled state in which the expandable bladder is configured to hold the biopharmaceutical composition therein;
a sensor system secured to an exterior surface of the bladder, the sensor system configured to transmit a signal indicative of a temperature of the exterior surface of the bladder; and
a processing device configured to receive the signal from the sensor system, the processing device providing a temperature of the biopharmaceutical composition at a desired location within the bladder based on the temperature of the exterior surface of the bladder.

11. The system according to claim 10, further comprising a probe system including a probe disposed within an interior of the expandable bladder, the probe configured to directly measure and transmit a signal indicative of a temperature at the desired location within the bladder.

12. The system according to claim 11, wherein the processing device is configured to correlate the signal provided by the sensor system to the temperature of the biopharmaceutical composition at the desired location within the bladder based on the signal of the probe.

13. A method of correlating an external sensor system for a storage container filled with a biopharmaceutical composition, the method comprising:
freezing the storage container filled with the biopharmaceutical composition;
generating a first temperature signal indicative of a temperature of an exterior surface of the storage container with the external sensor system secured to the exterior surface of the storage container during freezing of the storage container;
generating a second temperature signal indicative of a temperature of the biopharmaceutical composition within the storage container with an internal probe system disposed within an interior of the storage container in direct contact with the biopharmaceutical composition during freezing of the storage container;
receiving the first temperature signal and the second temperature signal with a processing device; and
generating correlated external temperature data to correlate the temperature of the exterior surface of the storage container to a temperature of the biopharmaceutical composition within the interior of the storage container based on the first temperature signal and the second temperature signal.

14. The method according to claim 13, further comprising freezing another storage container filled with the biopharmaceutical composition and determining a temperature of the biopharmaceutical composition within the other storage container with only an external sensor system using the correlated external temperature data.

15. The method according to claim 14, wherein determining the temperature of the biopharmaceutical composition within the other storage container includes looking up the temperature of the biopharmaceutical composition in a look up table of the correlated external temperature data.

16. The method according to claim 14, wherein determining the temperature of the biopharmaceutical composition within the other storage container includes calculating the temperature of the biopharmaceutical composition using a correlation algorithm of the correlated external temperature data.

17. The method according to claim 14, further comprising shipping the other storage container with the external sensor system secured to the storage container, the external sensor system disposed within extremities of a frame of the other storage container.

18. The method according to claim 13, wherein generating the second temperature signal includes the internal probe system disposed at a desired location within the interior of the storage container.

19. The method according to claim 13, further comprising inserting a probe of the probe system into an interior of the storage container such that a tip of the probe is positioned at a desired location within the interior of the storage container.

20. A method of correlating an external sensor system for a storage container filled with a biopharmaceutical composition, the method comprising:
freezing the storage container filled with the biopharmaceutical composition;
generating a temperature signal indicative of a temperature of an exterior surface of the storage container with the external sensor system secured to the exterior surface of the storage container during freezing of the storage container; and
determining a temperature of the biopharmaceutical composition at a desired location within the storage container using the temperature signal and the correlated external temperature data.
